(19) United States
(12) Patent Application Publication
(10) Pub. No.: US 2024/0366875 A1
(43) Pub. Date: Nov. 7, 2024

(54) FLUID MIXING DEVICES AND METHODS

(71) Applicant: Boston Scientific Scimed, Inc., Maple Grove, MN (US)

(72) Inventors: Mazen YATIM, Southborough, MA (US); Samuel GOSHEN, Brookline, MA (US); Kevin L. BAGLEY, Natick, MA (US)

(73) Assignee: Boston Scientific Scimed, Inc., Maple Grove, MN (US)

(21) Appl. No.: 18/650,193

(22) Filed: Apr. 30, 2024

Related U.S. Application Data
(60) Provisional application No. 63/500,008, filed on May 4, 2023.

(51) Int. Cl. A61M 5/28; A61M 5/00

(52) U.S. Cl. CPC A61M 5/284; A61M 2005/006

(57) ABSTRACT

A medical device may comprise: a first inlet for receiving a first fluid; a second inlet for receiving a second fluid; and a mixing body including a first body defining a first lumen and a second body disposed within the first lumen. The first lumen may be in fluid communication with the first inlet such that the first lumen receives the first fluid from the first inlet. The second body may define a second lumen. The second lumen may be in fluid communication with the second inlet, such that the second lumen receives the second fluid from the second inlet. A radially outer surface of the second body may have a plurality of openings, such that the first lumen and the second lumen are in fluid communication via the plurality of openings.

### CROSS-REFERENCE TO RELATED APPLICATIONS

This application claims the benefit of priority under 35 U.S.C. § 119 from U.S. Provisional Application No. 63/500,008, filed May 4, 2023, which is incorporated by reference herein in its entirety.

### TECHNICAL FIELD

The disclosure relates generally to fluid mixing devices and methods. More specifically, aspects of the disclosure pertain to medical devices and/or methods for combining a liquid with a fluid so as to aerate the liquid and facilitate delivery of the liquid to a target site in a subject's anatomy.

### BACKGROUND

Medical procedures may involve delivering a fluid, such as a liquid, to a site (e.g., a treatment site) within a subject's anatomy. For example, endoscopic procedures may be performed within a body lumen of a subject. Such procedures include endoscopic submucosal dissection (ESD) and endoscopic mucosal resection (EMR), which may be used to resect lesions (e.g., polyps) from a subject's body lumen (e.g., a colon). Perforations or other sources of bleeds may be a complication of such procedures. Bleeds may occur during a procedure or may be delayed. Low viscosity agents may be used prophylactically or hemostatically. Some low viscosity fluids may migrate from a site upon delivery due to their low viscosity. High viscosity agents pose deployment challenges due to an amount of force required to deliver a high viscosity liquid. Therefore, a need exists for delivering agents to a target site in a subject's anatomy.

### SUMMARY

This disclosure includes fluid mixing devices and methods of use thereof, e.g., devices and methods for mixing a high viscosity fluid with air, carbon dioxide, or another fluid, thereby facilitating flow of the fluid. Each of the aspects disclosed herein may include one or more of the features described in connection with any of the other disclosed aspects.

In aspects, a medical device may comprise: a first inlet for receiving a first fluid; a second inlet for receiving a second fluid; and a mixing body including a first body defining a first lumen and a second body disposed within the first lumen. The first lumen may be in fluid communication with the first inlet such that the first lumen receives the first fluid from the first inlet. The second body may define a second lumen. The second lumen may be in fluid communication with the second inlet, such that the second lumen receives the second fluid from the second inlet. A radially outer surface of the second body may have a plurality of openings, such that the first lumen and the second lumen are in fluid communication via the plurality of openings.

Any of the aspects disclosed herein may include any of the following features in any combination. The first body may be tubular. The second body may be tubular. The first body and the second body may be monolithically formed from a single piece of material. The first lumen may be coaxial with the second lumen. The mixing body may be sized so as to be received within a working channel of a scope. The first fluid may be a liquid, and the second fluid may be a gas. The mixing body may be configured such that the second fluid flows into the first lumen through the plurality of openings, thereby forming a combination of the first fluid and the second fluid within the first lumen. The medical device may further comprise an outlet that is configured to receive the combination of the first fluid and the second fluid from the first lumen. The first fluid may be a gas and the second fluid may be a liquid. The medical device may further comprise an outer sheath defining the first inlet; and an inner sheath at least partially within the outer sheath and defining the second inlet. A radially outer wall of the outer sheath may include a side opening. The inner sheath may be configured to receive the second fluid through the side opening. A proximal end of the first body may be coupled to a distal end of the outer sheath. A proximal end of the second body may be coupled to a distal end of the inner sheath. The mixing body may be configured such that the first fluid flows into the second lumen through the plurality of openings, thereby forming a combination of the first fluid and the second fluid within the second lumen. The mixing body may be configured such that a gas passes through the plurality of openings so as to form a combination of a gas and a liquid.

In another aspect, a medical device may comprise: a first body defining a first lumen; and a second body disposed within the first lumen. The second body may define a second lumen. A radially outer surface of the second body may have a plurality of openings, such that the first lumen and the second lumen are in fluid communication via the plurality of openings. The medical device may be configured such that a gas passes through the plurality of openings (a) from the first lumen into the second lumen or (b) from the second lumen into the first lumen, in order to form a combination of the gas and a liquid.

Any of the aspects disclosed herein may have any of the features below, in any combination. The first lumen may be coaxial with the second lumen. The first body may be tubular. The second body may be tubular.

In an aspect, a medical method may comprise: receiving a gas in a first lumen of a medical device; receiving a liquid in a second lumen of the medical device; passing the gas through a plurality of openings in a wall between the first lumen and the second lumen so that the gas combines with the liquid to form a combination of the gas and the liquid; and delivering the combination of the gas and the liquid to a location within a body lumen of a subject.

Any of the aspects disclosed herein may include any of the following steps or features. The passing the gas through the plurality of openings may disperse the liquid.

Any of the examples described herein may have any of these features in any combination.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a side view of a mixing device.

FIG. 2 depicts a cross-sectional side view of the mixing device of FIG. 1.

FIGS. 3 and 4 depict perspective cross-sectional views of the mixing device of FIG. 1.

FIG. 5 depicts another mixing device.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In certain figures, arrows labeled “P” and “D” denote proximal and distal directions, respectively. The term “exemplary” is used in the sense of “example,” rather than “ideal.” The term “approximately,” or like terms (e.g., “substantially”), includes values+/−10% of a stated value.

A medical device may be a mixing device that receives a first fluid, such as a treatment agent (e.g., a viscous liquid), and a second fluid, such as a gas (e.g., air or carbon dioxide). The medical device may slowly introduce the second fluid into the first fluid, thereby creating bubbles in the first fluid. The bubbles may expand, providing for increased dispersion/atomization of the first fluid. Thus, less pressure may be required in order to expel the first fluid (combined with the second fluid) from the medical device. In at least some examples, the first fluid (after mixing with the second fluid) may be delivered to a treatment site via an introducing device (e.g., a scope). In some examples, the first fluid may be mixed with the second fluid near a distal end of the introducing device, close to a treatment site. In other examples, the first fluid may be mixed with the second fluid near a proximal end of the introducing device (e.g., outside of a body lumen in which the introducing device is positioned), and the combined fluids may be advanced to the distal end of the introducing device and exit the introducing device to the treatment site. The devices described herein may be used to deliver agents, including viscous agents, fluid mixing devices and methods such as medical devices, and/or methods for delivering combining a liquid (e.g., a high viscosity liquid) with a fluid so as to aerate the liquid and facilitate delivery of the liquid, to a treatment site.

The combined first and second fluids may be delivered, via the introducing device, to a site of a procedure, such as EMR or ESD (e.g., to a site where a polyp has bene removed). The first fluid, without the second fluid, may otherwise not be deliverable hydraulically via the introducing device or require very high levels of delivery pressure. In some examples, combined fluid may be sprayed or otherwise may be administered to a treatment location. The mixing device and the combined fluid may be used in conjunction with materials that will solidify upon contact with tissue, adhere to the tissue, and cover a defect, thereby providing protection from delayed perforations and/or delayed bleeding.

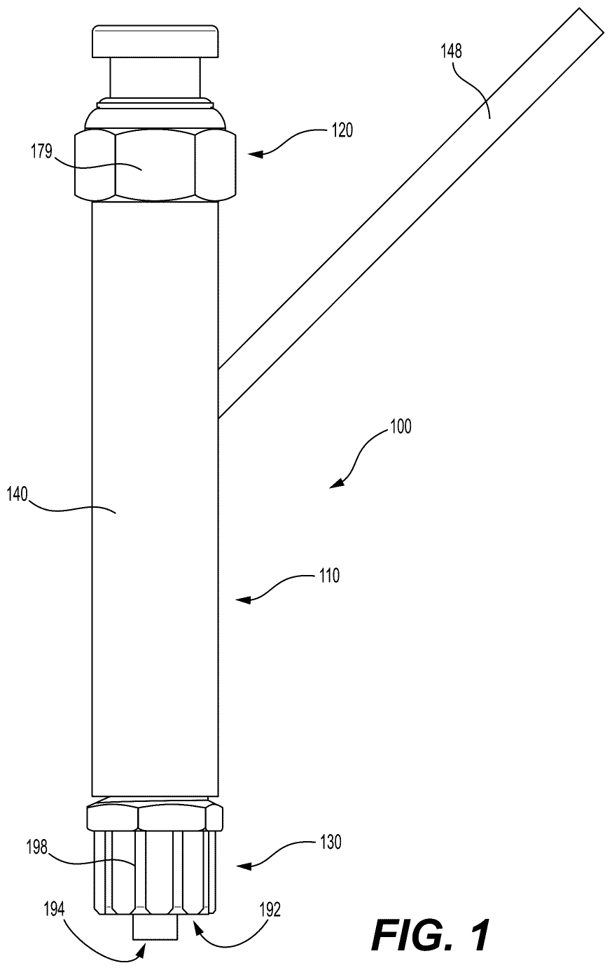

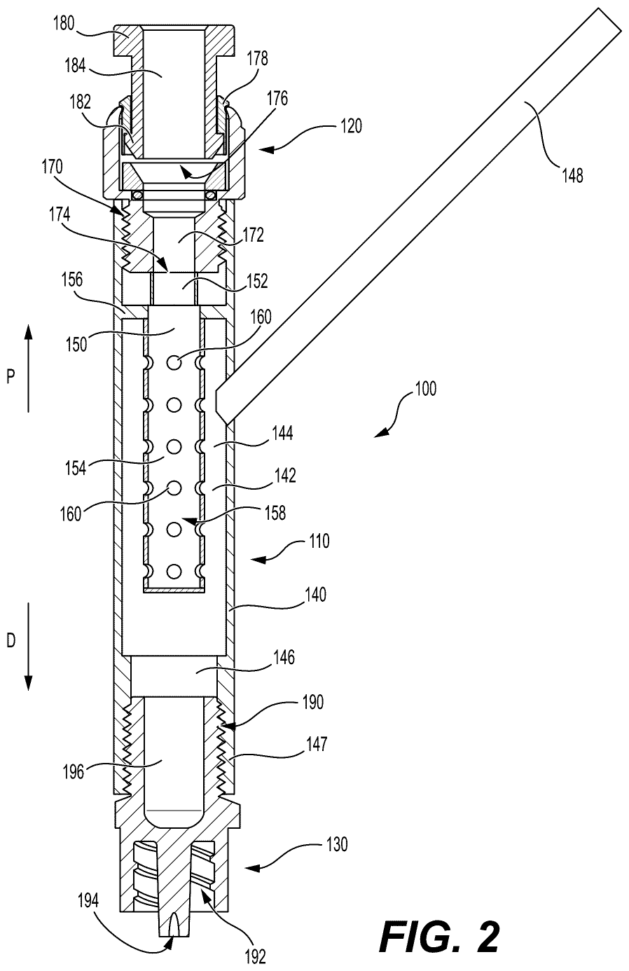

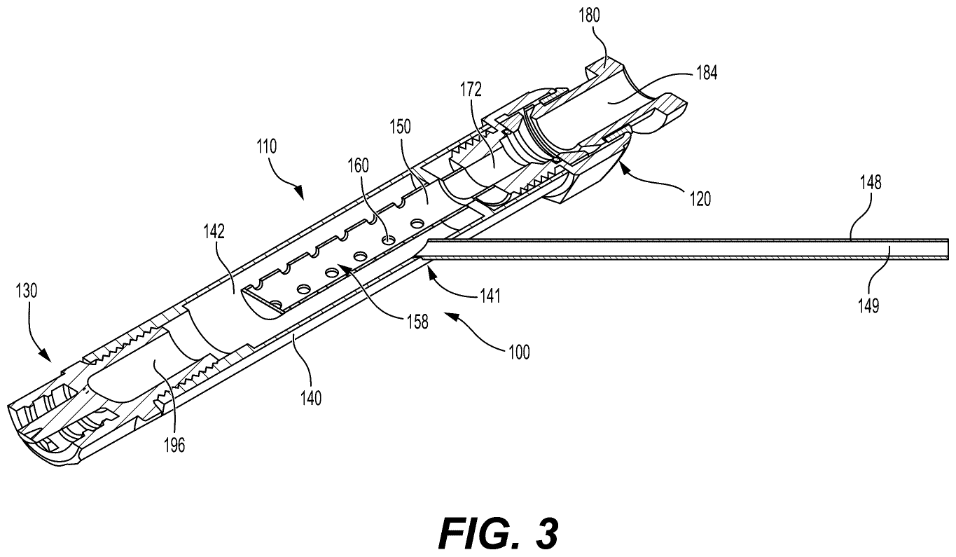

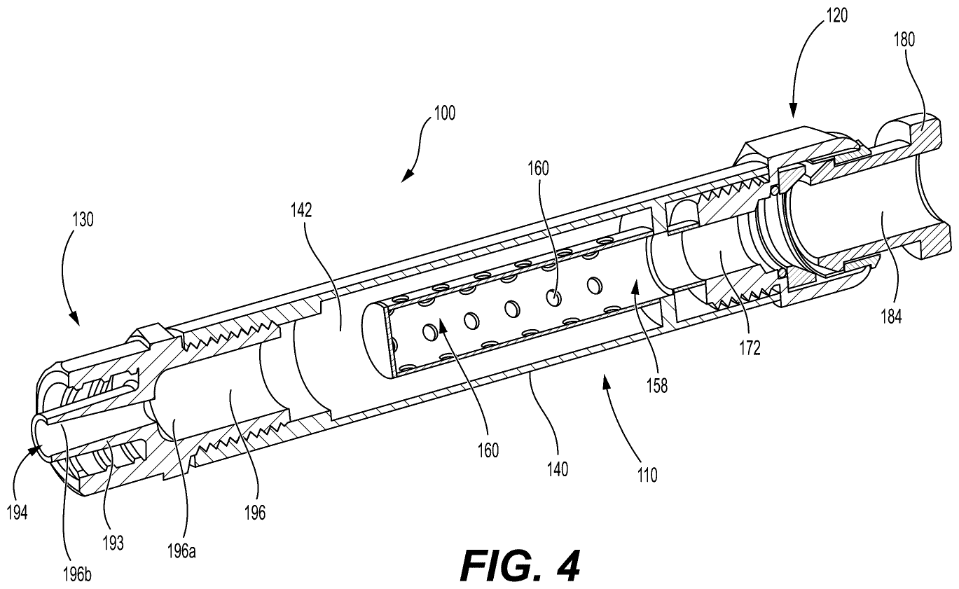

FIGS. 1-4 depict aspects of a medical device 100 that may be a mixing device used to combine a first fluid (e.g., a liquid treatment agent) with a second fluid (e.g., a gas, such as air or carbon dioxide). FIG. 1 depicts a side view of the medical device 100. FIGS. 2-4 depict a cross-sectional views of medical device 100, with the cross-section taken along/parallel to a longitudinal axis (proximal/distal axis) of medical device 100. FIG. 2 depicts a side view, while FIGS. 3 and 4 depict perspective views. It will be noted that the planes along which the cross-sections of FIGS. 2-4 are taken may differ from one another (but all may be parallel to a longitudinal axis of medical device 100). Thus, the visible features of FIGS. 2-4 may differ.

Medical device 100 may include a mixing body 110, an input body 120, and an output body 130. These bodies 110, 120, 130 may be separate components that are assembled together or an integral one-piece construction. The first fluid and the second fluid may be combined in mixing body 110. Input body 120 may channel (i.e., move) the second fluid into mixing body 110. Output body 130 may receive the combined first fluid and second fluid and may convey the combined first fluid and second fluid to another component (e.g., a catheter or distal tip, as described below). Medical device 100 also may include a first fluid conduit 148 for conveying the first fluid to the mixing body 110. Fluid conduit 148 may be integral with mixing body 110.

Mixing body 110 may include a housing 140. Housing 140 may have a generally tubular body having a cylindrical outer surface, as shown in FIG. 1. As shown in FIGS. 2-4, wall(s) of housing 140 may define a lumen 142. A cross-section of lumen 142 that is perpendicular to a longitudinal axis of mixing body 110 may have an approximately round shape. As shown particularly in FIG. 2, lumen 142 may include a proximal portion 144 and a distal portion 146. Proximal portion 144 may have a greater width/diameter than distal portion 146. However, such a configuration is merely exemplary, and lumen 142 may have a uniform diameter/width or may have other configurations having varying diameters/widths.

First fluid conduit 148 may extend from a side surface of mixing body 110 and may serve as an inlet for a first fluid. For example, as shown in FIGS. 1-4, first fluid conduit 148 may extend from mixing body 110 at an angle between zero degrees and 90 degrees relative to a longitudinal axis of mixing body 110. Alternatively, first fluid conduit 148 may extend at an angle of approximately 90 degrees relative to a longitudinal axis of mixing body 110 or at any other suitable angle. First fluid conduit 148 may be flexible or rigid. First fluid conduit may be tubular and define a lumen 149. Lumen 149 of first fluid conduit 148 may be in fluid communication with lumen 142 of mixing body 110 via, for example, an opening 141 (see FIG. 3) in a side wall of housing 140, such that fluid is received within lumen 142 via first fluid conduit 148. Due to the cross-section depicted in FIG. 4, first fluid conduit 148 is not visible in FIG. 4.

A second fluid conduit 150 may be a body disposed within housing 140. For example, second fluid conduit 150 may extend approximately parallel to a longitudinal axis of mixing body 110 (e.g., approximately coaxial with a central longitudinal axis of mixing body 110), through lumen 142. A cross-section of second fluid conduit 150 taken perpendicularly to a longitudinal axis of second fluid conduit 150 may be approximately circular. Portions of or an entirety of second fluid conduit 150 may have a tubular/annular/cylindrical shape. Second fluid conduit 150 may have a proximal portion 152 and a distal portion 154. Distal portion 154 may have a larger width/diameter than proximal portion 152. Alternatively, proximal portion 152 and distal portion 154 may have the same diameter/width, or distal portion 154 may have a smaller diameter/width than proximal portion 152. A support 156 may couple second fluid conduit 150 to housing 140 of mixing body 110. For example, support 156 may be an annular wall extending between housing 140 and distal portion 154 of second fluid conduit 150.

A lumen 158 may extend longitudinally through second fluid conduit 150. A side wall (a wall facing radially outward relative to a longitudinal axis of lumen 158) of second fluid conduit 150 may have a plurality of openings 160 formed therein. Second fluid conduit 150 may include any suitable number of openings 160, and openings 160 may be of any suitable size or shape (e.g., round). A size of openings 160 may be chosen depending on properties (e.g., viscosity) of the first fluid (e.g., a liquid agent) and/or the second fluid (e.g., a gas). For example, openings 160 may be approximately 0.5 mm to approximately 2.0 mm in diameter. Openings 160 may be placed at regular or irregular intervals longitudinally along second fluid conduit 150 (e.g., along distal portion 154). Openings 160 also may be placed at regular or irregular intervals circumferentially around second fluid conduit 150 (e.g., around distal portion 154). Second fluid conduit 150 may be fenestrated with any suitable arrangement of openings 160. For example, as shown, second fluid conduit 150 may include four rows of openings 160 that are parallel to a longitudinal axis of second fluid conduit 150. The four rows may be evenly spaced apart around a circumference of second fluid conduit 150 (i.e., 90 degrees apart). Each row may have six holes of the same size and shape. The fenestrated wall of second fluid conduit 150 may be disposed between/may form a boundary between lumen 158 and lumen 142. Openings 160 may fluidly couple lumen 158 of second fluid conduit 150 to lumen 142 of mixing body 110. A distalmost end of second fluid conduit 150 may be closed, so that the second fluid may flow only out of openings 160. Alternatively, the distalmost end of second fluid conduit 150 may be open. Second fluid conduit 150 may have a distalmost end that is proximal of a distalmost end of housing 140, and lumen 158 may have a distalmost end that is proximal of a distalmost end of lumen 142.

Input body 120 may be coupled (e.g., removably or fixedly coupled) to a proximal end of mixing body 110. Input body 120 and/or adapter 180 may be an inlet for the second fluid. Alternatively, input body 120 may be integrally formed with (i.e., formed in one single piece with) housing 140 of mixing body 110. Input body 120 may include threads 170 (see FIG. 2) for mating with corresponding threads of housing 140 to couple input body 120 to housing 140. As shown in FIG. 1, an outer surface of input body 120 may include a grip 179 (e.g., a ridged surface) for gripping and screwing input body 120 to housing 140. Input body 120 may define a passage 172 having an outlet 174 and an inlet 176. Outlet 174 may be fluidly (and sealingly) coupled to lumen 158 of second fluid conduit 150, such that passage 172 is in fluid communication with lumen 158. A width/diameter of outlet 174 may be substantially the same as a width/diameter of a lumen of proximal portion 152 of second fluid conduit 150.

As shown particularly in FIG. 2, an adapter 180 may be coupled to input body 120. In some examples, adapter 180 may be fixedly disposed on a distal end of tubing (not shown) carrying the second fluid from a source of the second fluid (e.g., a source of gas, such as air or carbon dioxide). In other examples, adapter 180 may removably receive a distal end of tubing (not shown) carrying the second fluid from the source of the second fluid. Adapter 180 may include one or more barbs 182. In an example, barb 182 may extend circumferentially about a distal end of adapter 180. Alternatively barbs 182 may be discrete from one another. Barb(s) 182 may interact with one or more projections 178 of input body 120. Projection(s) 178 may extend radially inward from an outer wall of input body 120. Barb(s) 182 may be deformable such that barb(s) 182 is/are compressible as adapter 180 is inserted into input body 120 and moved distally passed projection(s) 178. Barb(s) 182 may have shape memory properties, or are otherwise biased, such that they expand radially outward once distal of projection(s) 178. Barb(s) 182 may interact with projection(s) 178 to retain adapter 180 within input body 120. In some examples, adapter 180 may be removed from input body 120 after it is inserted into input body 120. In other examples, adapter 180 may not be removable after it is inserted into input body 120. Adapter 180 may have a push-to-connect interface with input body 120. Alternatively or additionally, tubing (not shown) may push to connect to adapter 180.

Adapter 180 may have a lumen 184 extending longitudinally therethrough. Lumen 184 may be open on proximal and distal ends thereof. A distal end of lumen 184 may be coupled to inlet 176 of passage 172. Lumen 184 may have approximately the same diameter/width as inlet 176. Thus, lumen 184 may be in fluid communication with passage 172.

Output body 130 may be coupled (e.g., removably or fixedly coupled) to a distal end of mixing body 110. Alternatively, output body 130 may be integrally formed with (i.e., formed in one single piece with) housing 140 of mixing body 110. For example, housing 140 may include an annular wall 147 projecting distally of a distal end of lumen 142. Output body 130 may include threads 190 on an outer surface thereof for mating and coupling with threads on an inner surface of annular wall 147. As shown in FIG. 1, an outer surface of output body 130 may include grips 198 to assist in coupling output body 130 to mixing body 110.

Alternatively, any other suitable mechanism may be used to couple output body 130 to mixing body 110. A distal end of output body 130 may include an interface 192 for coupling output body 130 to a catheter or other type of tubing (e.g., rigid or flexible tubing). For example, interface 192 may be a Luer-type interface.

Output body 130 may have a distal outlet 194 in fluid communication with a passage 196. Because a cross-section of FIGS. 2 and 3 differs from a cross-section of FIG. 4, a fluid connection between passage 196 and outlet 194 may be visible in FIG. 4 but not in FIGS. 2 and 3. Passage 196 may have a proximal portion 196a and a distal portion 196b (FIG. 4). Distal portion 196b may have a smaller width/diameter than proximal portion 196a. A width/diameter of proximal portion 196a may be approximately the same as a width/diameter of distal portion 146 of lumen 142 Proximal portion 196a may be coupled to distal portion 146 of lumen 142, such that lumen 142 is in fluid communication with passage 196. A width/diameter of distal portion 196b may be configured to be compatible with requirements of interface 192. In examples, distal portion 196b may be within a nozzle 193 (see FIG. 4) that may fit within a catheter or other tubing coupled to interface 192.

Alternatively to output body 130, any type of dispensing tip may be coupled to a distal end of mixing body 110. For example, a dispensing tip may include threads thereon for coupling to threads of housing 140. Dispensing tip may include an outlet having any of the features of outlet 194.

In operation, a source of second fluid (e.g., a gas, such as air or carbon dioxide) may be coupled to input body 120 (e.g., via adapter 180). For example, the source of the second fluid may be a pressurized source (e.g., a canister of carbon dioxide or a supply of compressed air). A regulator (not shown) may be used to adjust a flow rate of the second fluid (e.g., based on a viscosity of the first fluid).

A source of first fluid may be coupled to first fluid conduit 148. For example, the source of the first fluid may be a syringe or other container of the first fluid. The source of the first fluid may be activated to transmit the first fluid into the mixing body 110 via the first fluid conduit 148. The first fluid and the second fluid may be introduced into mixing body 110 simultaneously. Alternatively, the source of the first fluid may be activated before the flow of the second fluid, or vice versa. The sources of the first fluid and the second fluid may, as discussed below, provide a force/pressure to cause the first fluid and second fluid to flow through mixing body 110 and to cause a combination of the first and the second fluids to flow out of outlet 194.

The first fluid may be received by lumen 142 of mixing body 110 via first fluid conduit 148, which is fluidly coupled to lumen 142, as described above. The second fluid may travel through the lumen 184 of adapter 180, through the passage 172 of input body 120, and into lumen 158 of second fluid conduit 150. The second fluid may pass through/flow out of openings 160 and/or a distal end opening of second fluid conduit 150. A size of openings 160 and a flow rate of the source of the second fluid may be used to control a rate at which the second fluid flows through openings 160. The second fluid may pass relatively slowly through openings 160, thereby creating bubbles in the first fluid. The bubbles may expand within the first fluid, causing greater dispersion of the first fluid, even if the first fluid has a high viscosity. The combined first fluid and second fluid may travel through passage 196 and out of outlet 194, as the source of the first fluid and the source of the second fluid continue to be activated, thereby exerting distal force/pressure on the combined first fluid and second fluid. As the combined first fluid and second fluid travels out of outlet 194, at least some of the bubbles formed by the second fluid may pop.

Because the first fluid has been combined with the second fluid, a given volume of the combined first fluid and second fluid contains less of the first fluid than an equivalent volume that has only the first fluid (and does not include the second fluid). In effect, the second fluid (e.g., the air or carbon dioxide) breaks up the first fluid (e.g., the liquid therapeutic agent). The first fluid is dispersed/atomized by the second fluid. Thus, less force/pressure is required to expel the combined first and second fluid through passage 196 and out of outlet 194 than would be required to expel the first fluid alone. The force/pressure required to move the combined first and second fluid through any tubing/catheter attached to a distal end of mixing device 100 would similarly be less than would be required for the first fluid alone.

In some examples, the mixing body 110 may be inserted into a working channel of a delivery device, such as a scope (e.g., endoscope, colonoscope, duodenoscope, cystoscope), catheter, sheath, or the like. Mixing body 110 may be sized and shaped so as to be received by such a working channel. Thus, the mixing body 110 is disposed within a body lumen of a subject. The first fluid conduit 148 and a conduit/tubing coupled to adapter 180 may extend proximally through the delivery device. The first fluid (e.g., a liquid treatment agent) may be primed in a catheter affixed to input body 120 and/or adapter 180 and extending proximally through the working channel of the introduction device. In such examples, the first fluid and the second fluid may be mixed together at or near the distal end of the delivery device, proximate to a location at which the first fluid is desired to be delivered at a predetermined flow rate (e.g., a site where a lesion has been resected). In such examples, a dispensing tip may be utilized instead of output body 130. Additionally, or alternatively, a spray component may be attached to medical device 100 to allow for spraying of the combined fluids.

In other examples, the mixing body 110 may be disposed outside of a body lumen of a subject, proximal of a handle of the delivery device. A conduit/tubing (e.g., catheter) coupled to outlet 194 may be inserted into the working channel of the delivery device and advanced to the site at which the first fluid is desired to be delivered. The combined first and second fluids may be delivered to the site via the conduit/tubing. Such an arrangement may be advantageous where the first fluid's viscosity renders it difficult to transmit the first fluid to a distal end of the delivery device prior to mixing.

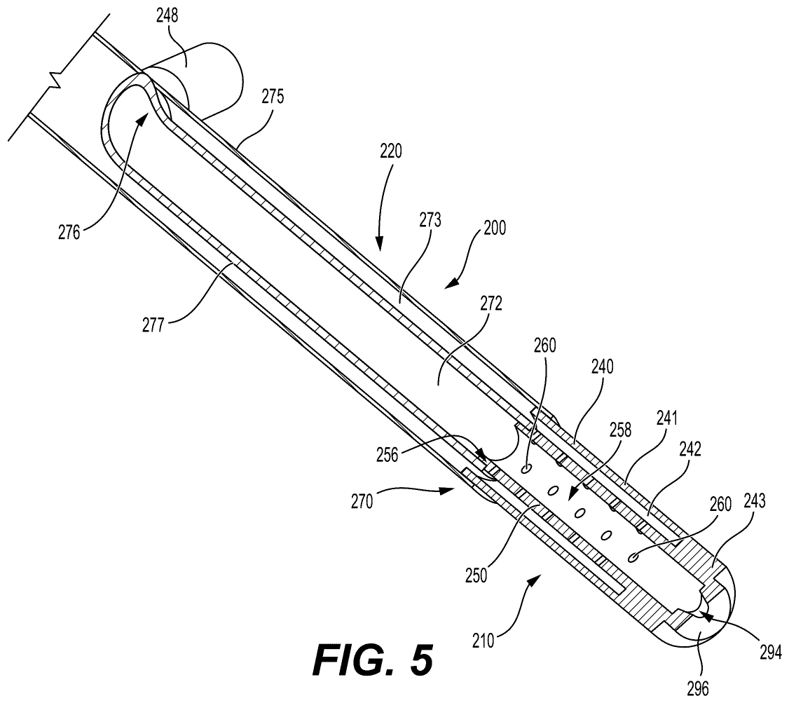

FIG. 5 depicts an alternative medical device 200 that may be used as a mixing device to combine a first fluid (e.g., a liquid treatment agent) with a second fluid (e.g., a gas, such as air or carbon dioxide). Unless otherwise specified below, medical device 200 may have any of the properties described above, with respect to medical device 100 and may function in any of the same manners with any of the same benefits. Aspects of medical device 100 and medical device 200 may be combined in any suitable manner.

Medical device 200 includes a mixing body 210 and a proximal portion 220 coupled to a proximal end of the mixing body 210. The first fluid and the second fluid may travel separately through the proximal portion 220 to the mixing body 210, and the first fluid and the second fluid are combined in the mixing body 210. The combined first and second fluids have any of the properties/features of the combined fluids discussed above, with respect to medical device 100.

Proximal portion 220 of medical device 200 may include an outer sheath 275 and an inner sheath 277. Inner sheath 277 may be at least partially disposed within outer sheath 275. Outer sheath 275 and inner sheath 277 may be bodies having any suitable properties. Outer sheath 275 and inner sheath 277 each may be tubular. In some examples, outer sheath 275 and inner sheath 277 each may be flexible catheters. In alternatives, portions of outer sheath 275 and/or inner sheath 277 may be rigid, or outer sheath 275 and/or inner sheath 277 may have varying flexibility along a length of the sheaths. Outer sheath 275 and inner sheath 277 may be approximately coaxial (i.e., a central longitudinal axis of outer sheath 275 may be approximately coaxial with a central longitudinal axis of inner sheath 277).

Each of outer sheath 275 and inner sheath 277 may be hollow, defining lumens, as discussed in further detail below. A gap between an inner wall of outer sheath 275 and an outer wall of inner sheath 277 may define a passage 273, which may be a portion of the lumen defined by the outer sheath. Passage 273 may have an annular cross-sectional shape. Passage 273 may be configured to carry the second fluid (e.g., a gas such as air or carbon dioxide). Inner sheath 277 may define a central lumen 272. Lumen 272 may be configured to carry the first fluid (e.g., a liquid treatment agent).

A first fluid inlet 248 may extend through a side wall of outer sheath 275 and may transmit a first fluid. First fluid inlet 248 may be sealingly coupled to a wall of outer sheath 275. For example, outer sheath 275 may include an opening through which first fluid inlet 248 passes, and walls of first fluid inlet 248 and outer sheath 275 may be sealingly coupled to one another. As shown in FIG. 5, first fluid inlet 248 may extend from outer sheath 275 at an approximately 90 degree angle. However, such an arrangement is merely exemplary, and first fluid inlet 248 may extend from outer sheath 275 at any desirable angle (e.g., an angle between 0 degrees and approximately 90 degrees). A lumen of first fluid inlet 248 may be in communication with lumen 272 via a junction 276 (e.g., an opening) formed in a side wall of inner sheath 277 and a distal end of first fluid inlet 248.

In some examples, first fluid inlet 248, outer sheath 275, and inner sheath 277 may be formed from a single, unitary piece of material (e.g., may be additively manufactured to form a single unitary body). In alternatives, one or more elements of first fluid inlet 248, outer sheath 275, and inner sheath 277 may be formed separately and coupled to one another. In an example, first fluid inlet 248 and inner sheath 277 may be formed form a single, unitary piece of material and may be coupled to outer sheath 275, which may be a separate piece of material. Such a configuration is merely exemplary, and any suitable arrangement may be utilized.

In some examples, first fluid inlet 248 and inner sheath 277 may form a single sheath (e.g., a single tubular structure). Such a combined sheath may bend at an angle (e.g., approximately 90 degrees or at any other suitable angle) and extend through a wall of outer sheath 275. In other examples, inner sheath 277 may continue proximally past first fluid inlet 248, through outer sheath 275. In such an example, lumen 272 may continue proximally past first fluid inlet 248 or may terminate at a proximal end of first fluid inlet 248 (e.g., portions of inner sheath 277 proximal of first fluid inlet 248 may have a solid cross-section and may not include a lumen). It will be appreciated that the exemplary structures discussed herein are not limiting and that any suitable structure may be employed so as to introduce the first fluid into the lumen 272.

A distal end of proximal portion 220 may be coupled to a proximal end of mixing body 210 at a junction 270. In some examples, elements of mixing body 210 may be formed from a single, unified (monolithic) piece of material. For example, mixing body 210 may be molded or additively manufactured to include all of the elements described herein. Alternatively, elements of mixing body 210 may be formed separately from one another and coupled together (fixedly or removably).

Mixing body 210 may include a housing 240. In some examples, a proximal end of housing 240 may be positioned within and coupled to a distal end of outer sheath 275. For example, housing 240 may be glued to outer sheath 275. Housing 240 may include an outer wall 241, which may have an annular shape in cross-section (i.e., a cylindrical/tubular shape).

An inner wall 250 may be disposed within outer wall 241. Inner wall 250 may be a body having an approximately annular shape in cross-section (i.e., a cylindrical/tubular shape) defining a lumen 258 with a central longitudinal axis (extending in a proximal/distal direction) that is approximately coaxial with a central longitudinal axis (extending in a proximal/distal direction) of housing 240. Inner wall 250 may have a tubular shape and may be flexible or rigid. As shown in FIG. 5, inner wall 250 may be formed integrally with outer wall 241, and both may be portions of housing 240. For example, inner wall 250 and outer wall 241 may be joined together at a distal wall 243. Alternatively, inner wall 250 may be a separate element (e.g., a flexible or rigid tube). Lumen 258 may be in fluid communication with (e.g., fluidly and/or sealingly coupled to) central lumen 272. Lumen 272 may be an inlet for lumen 258 through which fluid is transmitted, such that lumen 258 receives fluid from lumen 272. For example, a proximal end of inner wall 250 may be coupled (e.g., fixedly, sealingly coupled, via adhesive or another mechanism) to a distal end of inner sheath 277. For example, inner wall 250 may include a notch 256, and a distal end of inner sheath 277 may be received by and mate with notch 256.

A space between outer wall 241 and inner wall 250 may define a cavity (alternatively called a lumen/passage/compartment) 242. Cavity 242 may have an approximately annular shape. A proximal end of distal wall 243 may define a closed distal end of cavity 242. Cavity 242 may have an open proximal end (e.g., at a proximal end of mixing body 210/housing 240 and closed on a distal end, as described above). Cavity 242 may be in fluid communication with passage 273, such that cavity 242 receives fluid from passage 273. Passage 273 may be an inlet for cavity 242 through which fluid is transmitted. Although inner wall 250, housing 240, and distal wall 243 are described as separate elements, it will be appreciated that, as shown in FIG. 5, mixing body 210 may be described as a tubular element (housing 240) with an annular cavity 242 formed in an outer wall of the housing 240, extending from a proximal end of housing 240 and terminating proximally of a distal end of housing 240.

Inner wall 250 may face radially outward and may have a plurality of openings 260 formed therein. Openings 260 may have any property of openings 160, described above. In examples, openings 260 may extend along a longitudinal length (proximal/distal) of inner wall 250 and around a perimeter (e.g., circumference) of inner wall 250. Inner wall 250 may be fenestrated with any suitable pattern, such as the pattern described above with respect to openings 160. Inner wall 250 may form a boundary between cavity 242 and lumen 272. Via openings 260, cavity 242 may be in fluid communication with central lumen 272. A distalmost end of cavity 242 may be proximal of a distalmost end of central lumen 272.

A distal end of housing 240 may define a nozzle 296. Nozzle 296 may include an annular wall, which may have an outer circumference that is smaller than an outer circumference of outer wall 241. Nozzle 296 may define an outlet 294 having an open distal end. Outlet 294 may be in fluid communication with lumen 258 (e.g., may be distal of lumen 258). Outlet 294 may have a smaller width/diameter than lumen 258. Additionally or alternatively, medical device 200 may include an output body similar to output body 130, or housing 240 may be coupled to a dispensing tip (e.g., an off-the-shelf dispensing tip). Nozzle 296 may be directly coupled to mixing body 210 and may be integrally formed with mixing body 210. Thus, outlet 294 may be immediately adjacent to a distal end of lumen 258. Outlet 294 may optionally be coupled to a catheter or other element extending distally from outlet 294.

In use, medical device 200 may be coupled to sources of first fluid (e.g., a liquid treatment agent) and second fluid (e.g., a gas, such as air or carbon dioxide). Any of the sources of the first fluid and the second fluid, discussed above with respect to medical device 100, may be utilized. The first and second fluids may be delivered according to any of the mechanisms described above. A source of the first fluid may be coupled to first fluid inlet 248, and a source of the second fluid may be coupled to outer sheath 275, via any suitable mechanism. Medical device 200 may be inserted into a working channel of a delivery device (e.g., a scope) before or after the fluid sources are coupled to medical device 200. Medical device 200, including mixing body 210, may have dimensions (including a size and a shape) configured to be received within a working channel of the delivery device. Mixing body 210 (and nozzle 296) may be advanced to a distal end of the delivery device, to a procedure site within a subject's body lumen. In some examples, mixing body 210 may be at or near a distal opening of a working channel of the scope. First fluid inlet 248 (and a portion of device 200 coupled to a source of the second fluid) may remain outside of the subject's body (e.g., proximal of a handle of the scope).

The second fluid (e.g., a gas, such as air or carbon dioxide) may transmit/travel/flow distally to passage 273, and into cavity 242. The first fluid may transmit/travel/flow through, through lumen 272, and into lumen 258. The second fluid may pass into lumen 258 via passing through openings 260. The second fluid may mix with the first fluid, as described above, with respect to medical device 100. The combined first fluid and second fluid may exit through outlet 294 (i.e., be transmitted by outlet 294) and be dispensed at a location of a procedure within a subject's body lumen.

While principles of this disclosure are described herein with the reference to illustrative examples for particular applications, it should be understood that the disclosure is not limited thereto. Although aspects of the disclosure are described with respect to the examples described herein, it will be appreciated that features of the various examples may be combined in any suitable manner. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a first inlet for receiving a first fluid;
a second inlet for receiving a second fluid; and
a mixing body including:
a first body defining a first lumen, wherein the first lumen is in fluid communication with the first inlet such that the first lumen receives the first fluid from the first inlet; and
a second body disposed within the first lumen, wherein the second body defines a second lumen, wherein the second lumen is in fluid communication with the second inlet, such that the second lumen receives the second fluid from the second inlet, and wherein a radially outer surface of the second body has a plurality of openings, such that the first lumen and the second lumen are in fluid communication via the plurality of openings.

2. The medical device of claim 1, wherein the first body is tubular.

3. The medical device of claim 1, wherein the second body is tubular.

4. The medical device of claim 1, wherein the first body and the second body are monolithically formed from a single piece of material.

5. The medical device of claim 1, wherein the first lumen is coaxial with the second lumen.

6. The medical device of claim 1, wherein the mixing body is sized so as to be received within a working channel of a scope.

7. The medical device of claim 1, wherein the first fluid is a liquid, and the second fluid is a gas.

8. The medical device of claim 1, wherein the mixing body is configured such that the second fluid flows into the first lumen through the plurality of openings, thereby forming a combination of the first fluid and the second fluid within the first lumen.

9. The medical device of claim 8, further comprising an outlet that is configured to receive the combination of the first fluid and the second fluid from the first lumen.

10. The medical device of claim 1, wherein the first fluid is a gas and the second fluid is a liquid.

11. The medical device of claim 1, further comprising:
an outer sheath defining the first inlet; and
an inner sheath at least partially within the outer sheath and defining the second inlet.

12. The medical device of claim 11, wherein a radially outer wall of the outer sheath includes a side opening, and wherein the inner sheath is configured to receive the second fluid through the side opening.

13. The medical device of claim 11, wherein a proximal end of the first body is coupled to a distal end of the outer sheath, and wherein a proximal end of the second body is coupled to a distal end of the inner sheath.

14. The medical device of claim 1, wherein the mixing body is configured such that the first fluid flows into the second lumen through the plurality of openings, thereby forming a combination of the first fluid and the second fluid within the second lumen.

15. The medical device of claim 1, wherein the mixing body is configured such that a gas passes through the plurality of openings so as to form a combination of a gas and a liquid.

16. A medical device comprising:
a first body defining a first lumen; and
a second body disposed within the first lumen, wherein the second body defines a second lumen, and wherein a radially outer surface of the second body has a plurality of openings, such that the first lumen and the second lumen are in fluid communication via the plurality of openings;
wherein the medical device is configured such that a gas passes through the plurality of openings (a) from the first lumen into the second lumen or (b) from the second lumen into the first lumen, in order to form a combination of the gas and a liquid.

17. The medical device of claim 16, wherein the first lumen is coaxial with the second lumen.

18. The medical device of claim 17, wherein the first body is tubular, and wherein the second body is tubular.

19. A medical method, comprising:
receiving a gas in a first lumen of a medical device;
receiving a liquid in a second lumen of the medical device;
passing the gas through a plurality of openings in a wall between the first lumen and the second lumen so that the gas combines with the liquid to form a combination of the gas and the liquid; and
delivering the combination of the gas and the liquid to a location within a body lumen of a subject.

20. The medical method of claim 19, wherein the passing the gas through the plurality of openings disperses the liquid.
